(19) United States
(12) Patent Application Publication
(10) Pub. No.: US 2004/0081635 A1
(43) Pub. Date: Apr. 29, 2004

(54) Low dose IFN-gamma compositions and their use for treatment of interferon-sensitive diseases

(76) Inventors: Joseph M. Cummins, Amarillo, TX (US); Edward P. Amento, Portola Valley, CA (US)

Correspondence Address: BARNES & THORNBURG, 11 South Meridian Street, Indianapolis, IN, 46204, US

(21) Appl. No.: 10/691,210

(22) Filed: Oct. 22, 2003

Related U.S. Application Data
(63) Continuation of application No. 09/672,335, filed on Sep. 28, 2000, now abandoned.
(60) Provisional application No. 60/156,480, filed on Sep. 28, 1999.

(51) Int. Cl. A61K 38/21

(52) U.S. Cl. 424/85.6

(57) ABSTRACT

The use of low doses of IFN-gamma in the treatment of interferon-sensitive diseases is described. The IFN-gamma can be administered orally, preferably buccally or sublingually, or parenterally in low doses to activate monocyte, neutrophil, or B cell function, to decrease acute inflammation, or to treat cancer, bacterial or fungal diseases, or fibrosis in a patient suffering from such disease states. Pharmaceutical formulations containing low doses of IFN-gamma in combination with a pharmaceutical acceptable carrier and suitable for oral administration are also described.

### CROSS REFERENCE TO RELATED APPLICATIONS

This application claims priority under 35 U.S.C. § 119(e) to U.S. Provisional Application No. 60/156,480, filed Sep. 28, 1999 which is expressly incorporated by reference herein.

### FIELD OF THE INVENTION

The present invention relates to a composition and method for treatment of patients afflicted with IFN-gamma susceptible diseases. More particularly, this invention is directed to a low dose IFN-gamma pharmaceutical formulation and a method for using IFN-gamma for treatment of disease states selected from acute inflamation, monocyte and neutrophil dysfunction, attenuated B cell function, cancer, bacterial or fungal diseases, and fibrosis.

### BACKGROUND AND SUMMARY OF THE INVENTION

The prior art is replete with references to the use of interferons in the treatment of interferon-sensitive diseases. IFN-gamma is a commercially available pharmaceutical with but few FDA approved clinical indications. Treatment with gamma IFN is typically effected by administering doses of IFN-gamma ranging from about 10⁴ to 3×10⁶ IU of IFN-gamma/kg of patient body weight, typically administered via subcutaneous or intramuscular injection. While the oral ingestion and oral mucosal (sublingual or buccal) administration of low doses of alpha and beta interferon have been described in the art, there has been no description of the administration of very low doses of the notably acid-labile IFN-gamma compounds into the mouth for treatment of acute inflammation, for activation of monocyte, neutrophil and B-cell function, or for treatment of bacterial or fungal diseases as is described and claimed in accordance with the present invention. Nor is there any description of the oral administration of such low doses of gamma interferon for oncolytic or antifibrotic applications.

There still exists a significant need to optimize the efficiency of IFN-gamma administration and to continue diligent research and development work directed to discovery of new interferon administration protocols capable of eliciting heretofore unrecognized disease resisting immune responses.

The present invention is based in part on the discovery that IFN-gamma when administered into the oral cavity inhibits the egress of neutrophils from capillaries into an extra-vascular fluid space. The discovery was made during a study conducted in a model of acute inflamation in which a cavity is formed in subcutaneous tissue of mice by the repeated injection of air. A pro-inflammatory agent (IL-I mixed in carboxymethylcellulose (CMC)) is used to induce the acute influx of neutrophils into the blind cavity. Surprisingly, the study revealed that IFN-gamma administered to the oral cavity in low doses (10 IU per day for three days) to mice markedly inhibited neutrophil accumulation to a level equivalent to that obtained by administering 200,000 IU per day of IFN-gamma parenterally for three days. This observation is also predictive of the capacity of oral IFN-gamma to influence monocyte function. Neutrophils and monocytes are derived from a common precursor cell and many of the effects of IFN-gamma on neutrophils are also shared with monocytes. Thus, in accordance with another aspect of this invention low doses of IFN-gamma are administered into the oral cavity in an amount effective to enhance monocyte function and thereby benefit the treatment of infectious agents that reside primarily in monocytes, for example, tuberculosis and leprosy, and in treatment of chronic granulomatosis disease (CGD).

“Interferon” is a term generically encompassing a group of vertebrate glycoproteins and proteins which are known to have antiviral, antiproliferative and immunomodulatory activity. In the early years of interferon research, an international committee that was assembled to devise a system for orderly nomenclature of interferons defined “interferon” as follows: “To qualify as an interferon a factor must be a protein which exerts virus non-specific, antiviral activity at least in homologous cells through cellular metabolic process involving synthesis of both RNA and protein.” Journal of Interferon Research, 1, pp. vi (1980). “Interferon” as used herein in describing the present invention shall be deemed to have that definition and shall contemplate such proteins, including glycoproteins, regardless of their source or method of preparation or isolation.

Interferons have generally been named in terms of the species of animal cells producing the substance, the type of cell involved (e.g., leukocyte/lymphoblastoid or fibroblast) and, occasionally, the type of inducing material responsible for interferon production. The designations alpha (α), beta (β) and gamma (γ) have been used to correspond to the previous designations of leukocyte, fibroblast, and immune interferons, respectively. Alpha and beta interferons are usually acid-stable and correspond-to what have been called Type I interferons; gamma interferons are usually acid-labile and correspond to what have been called Type II interferons. More recently, interferon tau has been described as an interferon-alpha related Type I interferon produced by bovine and ovine trophoblasts.

Interferon of human and murine origin is quantified in the art in terms of International Units (IU). Interferons of other than human or murine origin can be used in accordance with this invention, and interferons isolated from native interferon-producing cell populations or from recombinant organisms can be used. In that presently accepted practices may not extend the use of “International Units” to quantify non-human and non-murine interferons, it shall be understood that administration of amounts of non-human/non-murine interferons having the same efficacy as the quantities (IU's) of human interferon specified in this description are within the scope of the present invention.

One embodiment of the present invention is directed to a method for reducing acute inflammation in a warm-blooded vertebrate suffering from such inflammation. The method comprises the steps of administering orally or parenterally to said vertebrate about 0.1 to about 10,000 IU of IFN-gamma/kg of body weight of said vertebrate and reducing said acute inflammation. The method comprises a step of administering a low dose (relative to art-recognized clinically acceptable parenteral doses of IFN-gamma) into the oral cavity of the vertebrate or parenterally. The orally administered IFN-gamma can be administered buccally, sublingually, pharyngeally, or by oral ingestion in solution or in a saliva-soluble dosage form. Acute inflammatory disease of the sort mediated by neutrophil ingress includes, but is not limited to, asthma or inflammation induced by radiation therapy for tumors in the lungs, brain or kidney, by reperfusion injury incident to stroke or coronary artery blockage, by traumatic injury to the brain or spinal cord, or by traumatic burns. It has also been found that intraperitoneal injection of low doses of IFN-gamma is significantly effective in treatment of acute inflammatory disease. Doses of IFN-gamma suitable for use in accordance with the present invention are those doses ranging from about 0.1 to about 10,000 IU of IFN-gamma/kilogram of body weight, and, more preferably, from about 1 to about 500 IU of IFN-gamma/kilogram of body weight, or about 1 to about 100 IU of IFN-gamma/kilogram of body weight.

In another embodiment of the invention a method is provided for treating or preventing IFN-gamma sensitive disease states selected from the group consisting of diseases characterized by monocyte and neutrophil dysfunction, cancer and fibrosis in a human patient suffering from such disease. The method comprises the steps of administering orally or parenterally to said patient about 0.1 to about 10,000 IU of IFN-gamma/kg of body weight of said vertebrate and treating or preventing said disease states. An antifibrotic or a chemotherapeutic agent known for use in cancer therapy or for treatment of immune diseases characterized by hypoactive or hyperactive immune system dysfunction may be used in combination with IFN-gamma in accordance with the present invention.

In another method embodiment of the present invention, IFN-gamma sensitive diseases, for example, chronic granulomatosis disease and osteopetrosis are treated by administering orally or parenterally about 0.1 to about 10,000 IU of IFN-gamma/kg of body weight of the vertebrate.

In yet another embodiment of the invention a method is provided for treating or preventing bacterial or fungal disease in a warm-blooded vertebrate susceptible to said diseases. The method comprises the steps of administering orally or parenterally to said vertebrate about 0.1 to about 10,000 IU of IFN-gamma/kg of body weight of said vertebrate and treating or preventing said bacterial or fungal disease.

In still another embodiment of the invention a method is provided for treating or preventing bacterial or fungal disease in a warm-blooded vertebrate susceptible to said diseases. The method comprises the steps of administering orally or parenterally to said vertebrate about 0.1 to about 10,000 IU of IFN-gamma/kg of body weight of said vertebrate and a therapeutic agent selected from the group consisting of an antibiotic and an antiflngal, and treating or preventing said bacterial or fungal disease.

The experimental data produced in the research work supporting the present invention also suggests that low doses of IFN-gamma activate B-cell populations. Accordingly, another aspect of this invention is a method of activating the B-cell population of a patient suffering from a disease state characterized by attenuated B-cell function. The method comprises the steps of administering orally or parenterally to said patient about 0.1 to about 10,000 IU of IFN-gamma/kg of body weight of said vertebrate and activating at least a portion of said B-cell population.

In still one other embodiment of the invention there is provided a pharmaceutical formulation for treatment of a disease selected from the group consisting of acute inflammation, monocyte, neutrophil, or B cell dysfunction, cancer, bacterial and fungal diseases, and fibrosis. The formulation comprising in unit dosage from about 10 to about 50,000 IU of human IFN-gamma and a pharmaceutically acceptable carrier therefor. The formulation can be in a liquid or solid form, and it is preferably formulated for administration to the oral cavity as a saliva-soluble solid. Optionally, the formulation can be in a lozenge dosage form for administration to a patient by holding the lozenge dosage form in the mouth to form a saliva solution of IFN-gamma in contact with the oral and pharyngeal mucosa. In combination with IFN-gamma, the formulations of the present invention may also comprise a therapeutic agent selected from the group consisting of an antibiotic, an antifungal, an antifibrotic, and a chemotherapeutic agent known for use in cancer therapy or for treatment of immune diseases characterized by hypoactive or hyperactive immune system dysfunction.

In other embodiments of the present invention IFN-gamma is administered to treat IFN-susceptible disease states in a human or other animal by its oral administration or parenteral administration at a dose of about 0.1 to about 5000 IU/kg, about 1 to about 1000 IU/kg, about 1 to about 500 IU/kg, about 1 to about 100 IU/kg, or about 0.1 to about 3 IU/kg of patient body weight.

### DETAILED DESCRIPTION OF THE INVENTION

The present invention is directed particularly to low dose IFN-gamma compositions and their use in methods for treatment of warm-blooded vertebrates with interferon-sensitive diseases. The present invention enables use of low doses of IFN-gamma of about 0.1 to about 10,000 IU of IFN-gamma/kg of body weight of the patient undergoing treatment. More preferably, in other embodiments of the invention the patient is treated with IFN-gamma at concentrations of about 0.1 to about 5000 IU/kg, about 1 to about 1000 IU/kg, about 1 to about 500 IU/kg, about 1 to about 100 IU/kg, or about 0.1 to about 3 IU/kg of patient body weight. The effective dose of the IFN-gamma can vary from species to species, and can depend on the condition of the animal being treated. The IFN-gamma may be administered into the oral cavity, most preferably sublingually or buccally, or may be administered parenterally. The interferon may also be administered by oral ingestion, intranasally, for example by inhalation of a powder or dispersed liquid droplets, or topically and may be administered in a pharmaceutically acceptable solid, liquid, or saliva-soluble dosage form (e.g., a lozenge).

The method of the present invention can be used for treatment of any warm-blooded vertebrate animal and is applicable to human clinical medicine and, potentially, to the treatment of agricultural animals, domestic animals, laboratory animals, or wild animals in captivity. Thus, the present invention has human clinical and veterinary applications among other applications. The present invention can be applied to warm-blooded vertebrate animals including, but not limited to, humans, laboratory animals such rodents (e.g., mice, rats, hamsters, etc.), rabbits, monkeys, chimpanzees, domestic animals such as dogs, cats, and rabbits, agricultural animals such as cows, horses, swine, sheep, goats, and poultry, and wild animals in captivity such as birds, bears, pandas, lions, tigers, leopards, elephants, zebras, giraffes, gorillas, bison, deer, antelope, marmosets, dolphins, whales, and any endangered animal.

The invention is applicable to such disease states as acute inflamation, monocyte and neutrophil dysfunction, attenuated B cell function, cancer, bacterial or fungal diseases, and fibrosis. The invention can be used to potentiate the immune response to cancers that are tumorigenic, including benign tumors and malignant tumors, or cancers that are non-tumorigenic. Such cancers may arise spontaneously or by such processes as mutations present in the germline of the host animal or somatic mutations, or may be chemically-, virally-, or radiation-induced. The invention can be utilized to enhance the immune response to such cancers as carcinomas, sarcomas, lymphomas, Hodgkin's disease, melanomas, mesotheliomas, Burkitt's lymphoma, nasopharyngeal carcinomas, leukemias, myelomas, and other neoplastic diseases. The cancer cell population can include, but is not limited to, oral, thyroid, endocrine, skin, gastric, esophageal, laryngeal, pancreatic, colon, bladder, bone, ovarian, cervical, uterine, breast, testicular, prostate, rectal, kidney, liver, and lung cancers.

The low doses of interferon-gama for use in accordance with the present invention can also be used to potentiate the immune response to exogenous pathogens or to a cell population harboring an endogenous pathogen, e.g., monocytes harboring tuberculosis or leprosy bacteria. The present invention is applicable to such exogenous pathogens as bacteria and fungi. Bacteria that may be treated with the present invention are any art-recognized bacteria that cause pathogenesis in warm-blooded vertebrate animals, including such organisms as bacteria that are gram-negative or gram-positive cocci or bacilli. Of particular interest are bacteria that are resistant to antibiotics such as antibiotic-resistant Streptococcus species and Staphlococcus species, or bacteria that are susceptible to antibiotics, but cause recurrent infections treated with antibiotics so that resistant organisms eventually develop. Such organisms can be treated with the low doses of IFN-gamma of the present invention in combination with lower doses of antibiotics than would normally be administered to warm-blooded vertebrates to avoid the development of these antibiotic-resistant bacterial strains.

The present invention is also applicable for use in enhancing the immune response to any fungi or mycoplasma species or other microorganisms that cause disease in warm-blooded vertebrate animals. Examples of fungi that may be treated with the method of the present invention include fungi that grow as molds or are yeastlike, including, for example, fungi that cause diseases such as ringworm, histoplasmosis, blastomycosis, aspergillosis, cryptococcosis, sporotrichosis, coccidioidomycosis, paracoccidiomycosis, and candidiasis.

Other afflictions responding to the low doses of IFN-gamma of the present invention are autoimmune disorders, inflammatory disorders, immunodeficiency disorders, and fibrosis. Low doses of IFN-gamma may be used to modulate the immune response to such autoimmune disorders as rheumatoid arthritis, systemic lupus erythematosus, multiple sclerosis, myasthenia gravis, Guillain-Barre syndrome, Graves disease, Sjogren's syndrome, autoimmune alopecia, scleroderma, psoriasis, and graft-versus-host disease.

Acute inflammatory disorders that respond to low doses of IFN-gamma in accordance with this invention include such hyperallergenic conditions as asthma, anaphylaxis, eczema, atopic and allergic contact dermatitis, and food, drug, and environmental allergies. Acute inflammation induced by radiation of the lungs, brain or kidney during radiation therapy for tumors, by reperfusion injury incident to stroke or coronary artery blockage, or by traumatic burns or traumatic injury to the brain or spinal cord also respond to the low doses of IFN-gamma of the present invention. Inflammatory disorders involving monocyte or neutrophil dysfunction that respond to low doses of interferon include such diseases as chronic granulomatous disease, Chediak-Higashi syndrome, Job's syndrome, systemic lupus erythematosus, osteopetrosis, and aplastic anemia.

Any immunodeficiency-related disorder, including disorders resulting from attenuated B cell function, may also be treated with the low doses of interferon of the present invention including such disorders as acquired immunodeficiency syndrome, xeroderma pigmentosa, severe combined immunodeficiencies, agammaglobulinemias, multiple myeloma, leukemias, and the like. Fibrosis, including interstitial joint and interstitial lung diseases and diseases of the lower bronchial or alveolar lining, may also be treated with the low doses of IFN-gamma used in accordance with this invention. Additionally, fibrotic diseases of any other organ or tissue, including the kidney, liver, heart, pericardium, retina or other ocular tissues, peritoneum, spinal tissue, and the meninges may be treated in accordance with this invention.

The method of the present invention may be used in combination with other therapies, such as in the case of treatment of cancer in combination with surgical removal of a tumor or radiation therapy or chemotherapy, or in the case of microbial diseases in combination with antibiotics. Antibiotics, including antimicrobials and antifungals, can be administered in combination with the low doses of IFN-gamma of the present invention. Such antibiotics include penicillins, cephalosporins, vancomycin, erythromycin, clindamycin, rifampin, chloramphenicol, aminoglycosides, gentamicin, and amphotericin B. In the case of fibrotic diseases, IFN-gamma may be used in combination with an antifibrotic.

IFN-gamma for administration into the oral cavity can be formulated utilizing art-recognized techniques into pharmaceutically acceptable liquid carriers, for example, in the form of a syrup, a suspension, or a spray, or they can be formulated in combination with pharmaceutically acceptable solid carriers in the form of tablets, capsules, caplets, gel-seals, lozenges or sachets.

The IFN-gamma intended for buccal, sublingual, or pharyngeal administration in accordance with the present invention is administered to the patient in a dosage form adapted to promote contact of the administered interferon with the patient's oral and pharyngeal mucosa. Thus, the dosage form can be formulated as an IFN-gamma-containing solution, including a suspension, a spray, or syrup, to be administered and used by the patient in a manner which promotes contact of the IFN-gamma component with the oral mucosal tissues, for example, by holding the interferon solution in the mouth for up to one or two minutes. Alternatively, the interferon can be administered by oral ingestion wherein the compounds are formulated into, for example, a syrup or a suspension to be swallowed by the patient and not held in the mouth. Syrups for either use may be flavored or unflavored and may be formulated using a buffered aqueous solution of interferon as a base with added caloric or non-caloric sweeteners, flavor oils and pharmaceutically acceptable surfactant/dispersants. Other liquid dosage forms known in the art can be prepared and can be administered buccally, sublingually, pharyngeally, or by oral ingestion. Alternatively, interferon may be administered into the stomach through a nasogastric tube and for the purposes of this invention such a route of administration is defined as oral administration.

Preferably, the IFN-gamma for use in the present invention is formulated into a solid dosage form comprising the low dose of IFN-gamma in a saliva-soluble carrier, optionally with desirable excipients, such as buffers or tableting aids. The solid dosage form is formulated to dissolve, when held in a patient's mouth, to form a saliva solution of the dose of IFN-gamma to promote contact of the interferon with the oral and pharyngeal mucosa.

Exemplary of saliva-soluble dosage forms are lozenges, tablets, caplets, capsules, gel-sols, sachets, and the like. In one embodiment, the solid dosage form is in the form of a lozenge adapted to be dissolved upon contact with saliva in the mouth, with or without assistance of chewing, to form a saliva solution of the interferon. Lozenge dosage forms of this invention can be prepared, for example, by art-recognized techniques for forming compressed tablets where the interferon is dispersed on a compressible solid carrier, optionally combined with any appropriate tableting aids such as a lubricant (e.g., magnesium stearate) and compressed into tablets. The solid carrier component for such tableting formulations can be a saliva-soluble solid, such as a cold-water-soluble starch or a monosaccharide or disaccharide, so that the lozenge will readily dissolve in the mouth to release the contained interferon in saliva solution for contact with and absorption by the oral/pharyngeal mucosa when the lozenge is held in the mouth. The pH of the above-described formulations can range from about 4 to about 8.5. Lozenges for use in accordance with the present invention can also be prepared utilizing other art-recognized solid unitary dosage formulation techniques.

Tablets for use in accordance with this invention can be prepared in a manner similar to that described for preparation of lozenges or by other art-recognized techniques for forming compressed tablets such as chewable vitamins. Suitable solid carrier components for tableting include manitol, microcrystalline cellulose, carboxymethyl cellulose, and dibasic calcium phosphate.

Solid dosage forms for oral ingestion administration include such dosage forms as caplets, capsules, and gel-seals. Such solid dosage forms can be prepared using standard tableting protocols and excipients to provide interferon gamma-containing capsules, caplets, or gel-seals. Any of the solid dosage forms for use in accordance with the invention, including lozenges and tablets, may be in a form adapted for sustained release of the IFN-gamma.

Parenteral dosage forms of IFN-gamma in accordance with this invention are typically in the form of a reconstitutable lyophilizate comprising the dose of IFN-gamma. The lyophilizate can be rehydrated using sterile saline, or another pharmaceutically-acceptable buffer optionally along with stabilizers known to those skilled in the art, for injection immediately prior to administration. Such parenteral administration may be intradermal, subcutaneous, intramuscular, intraperitoneal, intranasal, intravenous, or topical. Intranasally administered IFN-gamma may be administered in the form of, for example, a spray for inhalation of dispersed liquid droplets or a powder administered by inhalation.

Any topical dosage forms known to those skilled in the art may be used. For example, topical dosage forms may comprise IFN-gamma, and as stabilizers a trihydric or higher polyhydric sugar alcohol, an organic acid buffer, and a conventional pharmaceutical carrier or diluent. Optionally, the composition may further contain as a stabilizer a material such as an anionic surfactant, albumin, and combinations thereof. Exemplary of a trihydric or higher polyhydric sugar alcohol are glycerin, erythritol, sorbitol, mannitol, and the like. Organic buffers include buffers such as citrate, succinate, tartrate, fumarate, and acetate buffers.

A “pharmaceutical acceptable carrier” for use in accordance with the invention is compatible with other reagents in the pharmaceutical composition and is not deleterious to the patient. The pharmaceutically acceptable carrier formulations for pharmaceutical compositions adapted for oral ingestion or buccal/sublingual administration including lozenges, tablets, capsules, caplets, gel-seals, and liquid dosage forms, including syrups, sprays, and other liquid dosage forms, have been described above. IFN-gamma can also be adapted for parenteral administration in accordance with this invention using a pharmaceutical acceptable carrier adapted for use in a liquid dose form. Such a liquid solution of IFN-gamma may be in the form of a clarified solution or a suspension. Exemplary of a buffered solution suitable as a carrier of IFN-gamma administered parenterally in accordance with this invention is phosphate buffered saline prepared as follows:

A concentrated (20×) solution of phosphate buffered saline (PBS) is prepared by dissolving the following reagents in sufficient water to make 1,000 ml of solution: sodium chloride, 160 grams; potassium chloride, 4.0 grams; sodium hydrogen phosphate, 23 grams; potassium dihydrogen phosphate, 4.0 grams; and optionally phenol red powder, 0.4 grams. The solution is sterilized by autoclaving at 15 pounds of pressure for 15 minutes and is then diluted with additional water to a single strength concentration prior to use.

The pharmaceutical formulations in accordance with this invention may comprise about 10 to about 50,000 IU of IFN-gamma, more typically about 100 to about 10,000 IU of IFN-gamma in combiantion with a saliva-soluble carrier. The dose can be formulated using standard pharmaceutical formulation techniques for oral or parenteral administration with an acceptable carrier, alone or in combination with effective amounts of other therapeutic agents including antimicrobials, antifungals, antifibrotics, and chemotherapeutics known for use in cancer therapy and in treatment of autoimmune diseases characterized by hyperactive or hypoactive immune system dysfunction.

The daily doses of IFN-gamma for administration in accordance with the method of this invention can be administered as single doses, or they can be divided and administered as a multiple-dose daily regimen. Further, a staggered regimen, for example, one to three days' buccal/sublingual interferon treatments per week, can be used as an alternative to daily treatment, and for the purpose of defining this invention such intermittent or staggered daily regimen is considered to be equivalent to everyday treatment and within the scope of this invention. The IFN-gamma is administered in low doses one to three times per day until the symptoms of the IFN-sensitive disease have subsided. Typical periods for treatment vary significantly dependent on patient condition and the nature of the disease state. Also, effects similar to those produced by a given daily dosage administered for a given number of days can be achieved by administering lower dosages for a greater number of days, or a higher dosage for a smaller number of days.

### EXAMPLE 1

### Effect of Orally Administered Ifn-α and Systemically Administered IFN-Y On Neutrophil Activation

Methods

To form an air pouch the dorsal region of each mouse was shaved and wiped with alcohol. Sterile air (2.5 ml) was injected subcutaneously along the midline through a 0.2 μm syringe filter and a 30 gauge needle. As air was injected fingers were used to maintain symmetry and proper positioning of the air pouch. Three days later another 2.5 ml of sterile air was injected to further develop the pouch. After another three days, the proinflammatory agent was injected into the pouch.

The pro-inflammatory agent, IL-1 beta, was mixed with a 0.5% carboxymethylcellulose (CMC) solution in sterile PBS at a concentration of 40 ng/ml. IL-1 and 0.5 ml of a 40 ng/ml solution was injected into the air space of the pouch (30 gauge needle), and the pouch was gently massaged so that all areas of the pouch came into contact with the solution.

After 5 hours and 15 minutes, to collect neutrophils from the pouch, 2 ml of a washing solution (EDTA and heparin in PBS) was injected (18 gauge needle) into the air pouch, and the cellular contents of the pouch were removed. The pouch was washed thoroughly, and the fluid was collected using the same syringe. The samples were then centrifuged at 220×g for 15 minutes. Cells were resuspended with 2 ml of an EDTA-heparin solution in PBS and stained with Turks solution (10:1). Neutrophils were counted using a hemocytometer.

The interferons were diluted each day into 1×PBS and were administered orally by injecting 50 μl of an interferon solution into the mouth of each mouse using a plastic catheter attached to a 1 cc syringe. The interferons were injected once per day for three days (oral or I.P. administration). There were three mice per treatment group.

The data show the number of neutrophils per ml of fluid collected from each pouch. Dividing by the volume collected from each pouch slightly decreases the error but does not change any patterns. The percent difference is the percent decrease in polymorphonuclear leukocytes (PMN's) collected from pouches of mice treated with interferon relative to the untreated controls.

| | | % |
|---|---|---|
| | PMNs × 10⁴/ml | Difference |
| Treatment groups | | |
| IFN-alpha 1 IU orally 1× daily for 3 days | 56 | −44% |
| IFN-alpha 10 IU orally 1× daily for 3 days | 82 | −18% |
| Controls | | |
| PBS orally 1× daily for 3 days or I.P. as | 100 | |
| below | | |
| PBS orally 1× daily for 3 days, but no | 16 | −84% |
| IL-1 injected | | |
| IFN-gamma 2 × 10⁵ IU I.P. 1× daily 2 | 63 | −37% |
| days, 1 day, and 1 hour before | | |
| inflammation | | |

Conclusions

The results of this assay effectively demonstrate IL-1 induced acute inflammation. Significantly fewer neutrophils migrated into the air pouch where the mice were injected with CMC alone (as compared to mice injected with IL-1 and CMC). IFN-gamma injected I.P. (2×105 IU) reduced acute inflammation. This treatment can be used as a positive control in future experiments to ensure the validity of the assay. IFN-alpha administered orally (1-10 IU) also reduced acute inflammation.

### EXAMPLE 2

### Effect of Orally Administered IFN-α or IFN-Y On Neutrophil Activation

The protocols were similar to those described above for Example 1 except that a wider range of oral IFN-alpha doses were tested and a group of mice was treated with low doses of IFN-gamma administered orally. In addition, the mice were treated orally with the interferons three times daily rather than once daily, and neutrophils were collected four hours and 20 minutes after injections. There were eight mice in each group.

A Gilson pipettor was used to orally administer the interferon by pipetting 10 μl of IFN under the tongue; the small volume administered by pipetting was accurate and the solution was found to remain in the mouth.

| | | % |
|---|---|---|
| | PMNs × 10⁴/ml | Difference |
| Treatment groups | | |
| IFN-α 0.1 IU administered orally 3× | 41 | +24% |
| daily for 3 days | | |
| IFN-α 1 IU administered orally 3× | 36 | +9% |
| daily for 3 days | | |
| IFN-α 10 IU administered orally 3× | 32 | −3% |
| daily for 3 days | | |
| IFN-α 100 IU administered orally 3× | 35 | +6% |
| daily for 3 days | | |
| IFN-γ 10 IU administered orally 3× | 20 | −39%* |
| daily for 3 days | | P = 0.04 |
| IFN-γ 10 IU I.P. 1× daily 2 days, | 23 | −30% |
| 1 day, and 1 hour before inflammation | | |
| Controls | | |
| PBS orally as above or I.P. as above. | 33 | |
| PBS orally as above, but no IL-1 injected | 14 | −58%* |
| 2 × 10⁵ IFN-g I.P. as above. | 20 | −39%* |
| | | P = 0.03 |

Conclusions

Low-dose oral IFN-alpha given three times per day for three days at several concentrations did not reduce IL-1 induced neutrophil migration in this assay. However, low-dose (10 IU three times/day for three days) oral IFN-gamma significantly reduced neutrophil migration (39% reduction) and may be effective in reducing the severity of inflammation.

### EXAMPLE 3

### Effect of the Carrier on IFN-γ-Induced Inhibition of Neutrophil Recruitment

The protocols were similar to those described in Example 2 except that mice were treated with a range of IFN-γ doses administered orally. There were 5 mice per treatment group. The purpose of this study was to determine if adding a carrier protein to the IFN-gamma solutions was associated with reduced inflammation. Groups of mice treated with two different doses of IFN-gamma administered orally and a group treated with IFN-gamma injected I.P. were included. The vehicle used was 5% maltose and 0.1% bovine albumin in PBS.

| | | % |
|---|---|---|
| | PMNs × 10⁴/ml | Difference |
| Treatment groups | | |
| IFN-γ 10 IU 3× daily orally for 3 days | 95 | −20%* |
| | | P = 0.035 |
| IFN-γ 100 IU 1× daily orally for 3 days | 82 | −31%* |
| | | P = 0.016 |
| IFN-γ 1 × 10⁴ IU I.P. 1× daily 2 days, 1 | 52 | −56%* |
| day, and 1 hour before inflammation | | P = 0.0002 |
| Controls | | |
| Vehicle alone orally 3× daily for 3 days | 119 | |

Conclusions

In support of the findings of Experiment 2, 10 IU IFN-gamma administered orally three times per day for three days prior to inflammation significantly reduced neutrophil accumulation (20% reduction). The effect was even greater (31% reduction) with 100 IU of IFN-gamma administered orally three times per day for three days. Acute inflammation was also diminished (56% reduction) in the group treated systemically by injection of 1×10⁴ IU of IFN-gamma. Either the combination of maltose and albumin, or PBS, appear to protect the IFN-gamma equally well.

### EXAMPLE 4

### Effect of Higher Doses of Orally Administered IFN-γ On

The protocols were similar to those described in Example 2 except that the interferon treatment time was increased to six days, and interferon dilutions were made immediately before administration. A group of mice treated with 1000 IU of interferon was also included and nine-day-old pouches were inflamed instead of six-day-old pouches. There were ten mice per treatment group.

| | | % |
|---|---|---|
| | PMNs × 10⁴/ml | Difference |
| Treatment groups | | |
| IFN-γ 10 IU orally 3× daily for 6 days | 133 | 0% |
| IFN-γ 100 IU orally 3× daily for 6 days | 99 | −26% |
| IFN-γ 1000 IU orally 3× daily for 6 days | 91 | −31%* |
| | | P = 0.043 |
| Controls | | |
| Vehicle 3× orally daily for 6 days | 133 | |
| Vehicle 3× daily for 6 days, but no IL-1 | 33 | −75% |
| injected | | |
| IFN-γ 2 × 10⁴ IU I.P. 1× daily 2 days, | 124 | −7% |
| 1 day, | | |
| and 1 hour before inflammation | 84 | −37%* |
| (N = 5) | | P = 0.021 |

Conclusions

IFN-gamma 1000 IU administered orally three times per day for six days prior to inflammation significantly reduced neutrophil accumulation (31% reduction). An effect was also seen with 100 IU of oral IFN-gamma (26% reduction). I.P. injections with 2×10⁴ IU of IFN-gamma caused a significant reduction in neutrophil migration (37% reduction). Low doses of IFN-gamma administered orally were again effective in reducing accumulation of neutrophils at the site of acute inflammation. Thus, orally administered low-dose IFN-gamma and low-dose parenterally administered IFN-γ decrease the severity of acute inflammation as demonstrated by the murine air pouch model.

### EXAMPLE 5

### Effect of Orally Administered IFN-γ On the Acute and Ouiescent Phases of Murine Mycobacterium Tuberculosis Infection

A total of 360 female C57B46 mice susceptible to the Erdman strain of human Mycobacterium tuberculosis were used in a study to determine the biologic activity of human lymphoblastoid interferon alpha and gamma (HBL IFNα and IFNγ) administered by the oral mucosal route for the treatment of experimentally induced tuberculosis (TB) infection in mice.

Acute Phase:

Animals—180 mice Inoculation—study day 0

Therapy—study days −7 to +7, every other day (8 doses)

Treatment groups—IFNα and IFNγ, 90 mice each

Dosage Groups—0, 0.001, 0.01, 0.1, 1.0 and 10.0 IU, 15 mice each

Sample-study days 10, 20, and 30, 5 mice from each dosage group

Quiescent Phase:

Animals—180 mice Inoculation—study day 0

Therapy—study days 60 to 88, every other day (14 doses)

Treatment groups—IFNα and IFNγ, 90 mice each

Dosage Groups—0, 0.001, 0.01, 0.1, 1.0 and 10.0 IU, 15 mice each

Sample-study days 80, 100, and 120, 5 mice from each dosage group

Evaluations: The following endpoints will be used for efficacy.

CFU per gram of lung and spleen tissue were assayed. Analysis of variance for appropriate comparisons were performed on the CFU of each treatment group. The model system does not normally produce mortality in the mice; therefore, any premature deaths were recorded. A Chi-square test was used to identify statistical differences in mortality rates between appropriate groups.

Results of studies using IFNγ during the acute phase of the infection produced interesting results. At the 30-day sampling, the three highest doses of IFNγ (8 doses of 0.1, 1.0 and 10.0 IU every other day starting at study day—7), had significantly fewer (p=0.01) CFU than the control group.

What is claimed is:

1. A method for reducing acute inflammation in a warm-blooded vertebrate suffering from such inflammation, said method comprising the steps of administering orally or parenterally to said vertebrate about 0.1 to about 10,000 IU of IFN-gamma/kg of body weight of said vertebrate and reducing said acute inflammation.

2. The method of claim 1 wherein the IFN-gamma is administered buccally or sublingually in a solution or in a solid saliva-soluble dosage form.

3. The method of claim 1 wherein the vertebrate is a human patient suffering from an inflammation induced by radiation of the lungs, brain or kidney during radiation therapy for tumors.

4. The method of claim 1 wherein the acute inflammation is the result of reperfusion injury incident to stroke or coronary artery blockage.

5. The method of claim 1 wherein the warm-blooded vertebrate is a human patient suffering from a traumatic injury to the brain or spinal cord.

6. The method of claim 1 wherein the acute inflammation is the result of traumatic bums in a human patient.

7. The method of claim 1 wherein the acute inflammation is asthma.

8. The method of claim 1 wherein the interferon-gamma is administered at about 1 to about 500 IU of interferon-gamma/kg of body weight of said vertebrate.

9. The method of claim 1 wherein the interferon-gamma is administered at about 1 to about 100 IU of interferon-gamma/kg of body weight of said vertebrate.

10. A method for treating or preventing IFN-gamma sensitive disease states selected from the group consisting of diseases characterized by monocyte and neutrophil dysfunction, cancer and fibrosis in a human patient suffering from such disease, said method comprising the steps of administering orally or parenterally to said patient about 0.1 to about 10,000 IU of IFN-gamma/kg of body weight of said vertebrate, and treating or preventing said disease states.

11. The method of claim 10 wherein the disease state is selected from the group consisting of chronic granulomatosis disease and osteopetrosis.

12. The method of claim 10 wherein the disease state is fibrosis of any organ.

13. The method of claim 10 wherein the interferon-gamma is administered at about 1 to about 500 IU of interferon-gamma/kg of body weight of said vertebrate.

14. The method of claim 10 wherein the interferon-gamma is administered at about 1 to about 100 IU of interferon-gamma/kg of body weight of said vertebrate.

15. A method for treating or preventing bacterial or fungal disease in a warm-blooded vertebrate susceptible to said diseases comprising the steps of administering orally or parenterally to said vertebrate about 0.1 to about 10,000 IU of IFN-gamma/kg of body weight of said vertebrate and treating or preventing said bacterial or fungal disease.

16. The method of claim 15 wherein the IFN-gamma is administered into the oral cavity.

17. The method of claim 16 wherein the IFN-gamma is administered sublingually or buccally.

18. The method of claim 15 wherein the IFN-gamma is administered in a liquid dosage form.

19. The method of claim 15 wherein the IFN-gamma is administered in a solid dosage form.

20. The method of claim 19 wherein the solid dosage form is saliva-soluble and prepared for dissolution in saliva in the mouth.

21. The method of claim 15 wherein the interferon-gamma is administered at about 0.1 to about 5000 IU of interferon-gamma/kg of body weight of said vertebrate.

22. The method of claim 15 wherein the interferon-gamma is administered at about 1 to about 500 IU of interferon-gamma/kg of body weight of said vertebrate.

23. The method of claim 15 wherein the interferon-gamma is administered at about 1 to about 100 IU of interferon-gamma/kg of body weight of said vertebrate.

24. A pharmaceutical formulation for treatment of a disease selected from the group consisting of acute inflammation, monocyte, neutrophil, or B cell dysfunction, cancer, bacterial and fungal diseases, and fibrosis, said formulation comprising in unit dosage form about 10 to about 50,000 IU of human IFN-gamma and a pharmaceutically acceptable carrier therefor.

25. The pharmaceutical formulation of claim 24 in liquid form.

26. The pharmaceutical formulation of claim 24 in solid form.

27. The pharmaceutical formulation of claim 24 wherein the pharmaceutical acceptable carrier comprises a saliva-soluble solid and the formulation is in lozenge dosage form.

28. A pharmaceutical formulation for treatment of a disease selected from the group consisting of acute inflammation, monocyte, neutrophil, or B cell dysfunction, cancer, bacterial and fungal diseases, and fibrosis, said formulation comprising in unit dosage form about 10 to about 50,000 IU of human IFN-gamma, a therapeutic agent selected from the group consisting of an antibiotic, an antifungal, an antifibrotic, and a chemotherapeutic agent known for use in cancer therapy or for treatment of immune diseases characterized by hypoactive or hyperactive immune system dysfunction, and a pharmaceutically acceptable carrier therefor.

29. A method of activating the B-cell population of a patient suffering from a disease state characterized by attenuated B-cell function said method comprising the steps of administering orally or parenterally to said patient about 0.1 to about 10,000 IU of IFN-gamma/kg of body weight of said vertebrate and activating at least a portion of said B-cell population.

30. The method of claim 29 wherein the IFN-gamma is administered into the oral cavity.

31. The method of claim 30 wherein the IFN-gamma is administered sublingually or buccally.

32. The method of claim 29 wherein the IFN-gamma is administered in a liquid dosage form.

33. The method of claim 29 wherein the IFN-gamma is administered in a solid dosage form.

34. The method of claim 33 wherein the solid dosage form is saliva-soluble and is in lozenge dosage form.

35. The method of claim 29 wherein the interferon-gamma is administered at about 0.1 to about 5000 IU of interferon-gamma/kg of body weight of said vertebrate.

36. The method of claim 29 wherein the interferon-gamma is administered at about 1 to about 500 IU of interferon-gamma/kg of body weight of said vertebrate.

37. The method of claim 29 wherein the interferon-gamma is administered at about 1 to about 100 IU of interferon-gamma/kg of body weight of said vertebrate.

38. A method for treating or preventing bacterial or fungal disease in a warm-blooded vertebrate susceptible to said diseases, the method comprising the steps of administering orally or parenterally to said vertebrate about 0.1 to about 10,000 IU of IFN-gamma/kg of body weight of said vertebrate and a therapeutic agent selected from the group consisting of an antibiotic and an antifungal, and treating or preventing said bacterial or fungal disease.

40. A method for treating or preventing IFN-gamma sensitive disease states selected from the group consisting of diseases characterized by monocyte and neutrophil dysfunction, cancer and fibrosis in a human patient suffering from such disease, said method comprising the steps of administering orally or parenterally to said patient about 0.1 to about 10,000 IU of IFN-gamma/kg of body weight of said vertebrate and a therapeutic agent selected from the group consisting of an antifibrotic and a chemotherapeutic agent known for use in cancer therapy or for treatment of immune diseases characterized by hypoactive or hyperactive immune system dysfunction, and treating or preventing said disease states.
